(19) United States
(12) Patent Application Publication
(10) Pub. No.: US 2022/0168304 A1
(43) Pub. Date: Jun. 2, 2022

(54) METHODS FOR TREATING CYSTIC FIBROSIS

(71) Applicant: CHILDREN'S HOSPITAL MEDICAL CENTER, Cincinnati, OH (US)

(72) Inventors: Anil Goud JEGGA, West Chester, OH (US); Anjaparavanda P. NAREN, Cincinnati, OH (US); Kavisha ARORA, Cincinnati, OH (US)

(73) Assignee: CHILDREN'S HOSPITAL MEDICAL CENTER, Cincinnati, OH (US)

(21) Appl. No.: 17/650,846

(22) Filed: Feb. 12, 2022

Related U.S. Application Data
(63) Continuation of application No. 16/614,356, filed on Nov. 16, 2019, now abandoned.
(60) Provisional application No. 62/509,961, filed on May 23, 2017.

(51) Int. Cl. A61K 31/517; A61K 31/404; A61K 31/443; A61K 31/473

(52) U.S. Cl. CPC A61K 31/517; A61K 31/404; A61K 9/0075; A61K 31/473; A61K 31/443

(57) ABSTRACT

Some embodiments of the invention include methods for treating an animal for cystic fibrosis comprising one or more administrations of one or more compositions comprising saracatinib. Other embodiments of the invention include treating an animal N for cystic fibrosis comprising one or more administrations of one or more compositions comprising saracatinib, optionally a corrector of ΔF508 CFTR, and optionally a potentiator of ΔF508 CFTR. Still other embodiments of the invention include methods for treating a human with cystic fibrosis caused by the F508 deletion mutation in the cystic fibrosis transmembrane conductance regulator (CFTR), comprising one or more administrations of one or more compositions comprising saracatinib, and optionally VX770, VX809, or both. Additional embodiments of the invention are also discussed herein.

### CROSS-REFERENCE TO RELATED APPLICATIONS

This application claims the benefit of U.S. Provisional Application No. 62/509,961, filed May 23, 2017 entitled “Novel Compound as a Candidate Therapy for Cystic Fibrosis” which is herein incorporated by reference in its entirety.

### BACKGROUND

Cystic fibrosis (CF) is a genetic disorder that can affect several organs, including the lungs. Cystic fibrosis is an inherited disease and is caused by mutations in both copies of the gene for the cystic fibrosis transmembrane conductance regulator (CFTR) protein. When CFTR is not functional, several symptoms occur; for example, typically thin secretions (e.g., mucus and sweat) become thick. One CFTR mutation resulting in cystic fibrosis is the deletion of phenylalanine 508 (ΔF508); this mutation (ΔF508 CFTR) impairs CFTR folding, impairs CFTR biosynthetic and endocytic processing, and impairs CFTR chloride channel function. Attempts have been made to treat cystic fibrosis, but they are inadequate for a variety of reasons.

Thus, attempts to develop a clinically effective treatment of cystic fibrosis have been unsuccessful, and there is still a need to find treatments for cystic fibrosis.

Some embodiments of the invention include methods for treating an animal for cystic fibrosis comprising one or more administrations of one or more compositions comprising saracatinib. Other embodiments of the invention include treating an animal for cystic fibrosis comprising one or more administrations of one or more compositions comprising saracatinib, optionally a corrector of ΔF508 CFTR, and optionally a potentiator of ΔF508 CFTR. Still other embodiments of the invention include methods for treating a human with cystic fibrosis caused by the F508 deletion mutation in the cystic fibrosis transmembrane conductance regulator (CFTR), comprising one or more administrations of one or more compositions comprising saracatinib, and optionally VX770, VX809, or both. Additional embodiments of the invention are also discussed herein.

### SUMMARY

Some embodiments of the invention include a method for treating an animal for cystic fibrosis, comprising one or more administrations of one or more compositions comprising saracatinib, wherein the compositions may be the same or different if there is more than one administration. In other embodiments, at least one of the one or more compositions further comprises a corrector of ΔF508 CFTR, a potentiator of ΔF508 CFTR, or both. In yet other embodiments, at least one of the one or more compositions further comprises one or more of VX809, VX661, or VX770. In still other embodiments, at least one of the one or more compositions further comprises VX770. In certain embodiments, at least one of the one or more compositions further comprises VX809. In some embodiments, at least one of the one or more compositions further comprises VX661. In other embodiments, at least one of the one or more compositions further comprises VX770 and VX809.

In some embodiments, the amount of saracatinib in at least one of the one or more compositions is from about 0.0001% (by weight total composition) to about 99%. In other embodiments, the amount of saracatinib in at least one of the one or more compositions is no more than about 3.0 mg/kg animal weight. In certain embodiments, the amount of saracatinib in at least one of the one or more compositions is no more than about 2.0 mg/kg animal weight. In other embodiments, at least one of the one or more comp [symbol] a formulary ingredient.

In still other embodiments, at least one of the one or more compositions is a pharmaceutical composition. In certain embodiments, at least one of the one or more administrations comprises parenteral administration, a mucosal administration, intravenous administration, depot injection, subcutaneous administration, topical administration, intradermal administration, oral administration, sublingual administration, intranasal administration, or intramuscular administration. In still other embodiments, at least one of the one or more administrations comprises an intranasal administration, an aerosol administration, a nebulizer administration, a pressurized metered-dose inhaler (pMDI) administration, an inhaler administration, or a dry powder inhaler (DPI) administration. In yet other embodiments, if there is more than one administration at least one composition used for at least one administration is different from the composition of at least one other administration. In certain embodiments, one or more of VX809, VX661, or VX770 in at least one of the one or more compositions is administered to the animal in an amount of from about 0.005 mg/kg animal body weight to about 100 mg/kg animal body weight.

In some embodiments, the animal is a human, a rodent, or a primate. In other embodiments, the animal is in need of treatment of cystic fibrosis (e.g., cystic fibrosis caused by one or more mutations in the cystic fibrosis transmembrane conductance regulator (CFTR)). In certain embodiments, the method is for treating cystic fibrosis caused by one or more mutations in the cystic fibrosis transmembrane conductance regulator (CFTR). In certain embodiments, the method is for treating cystic fibrosis caused by the F508 deletion mutation in the cystic fibrosis transmembrane conductance regulator (CFTR)

In some embodiments, the method further comprises treatment of a cancer related to cystic fibrosis. In other embodiments, the method further comprises treatment of a cancer related to cystic fibrosis and the cancer related to cystic fibrosis is a lung cancer, a digestive tract cancer, colon cancer, cancer at the cardio-esophageal junction, esophageal cancer, cancer at the gastro-esophageal (or squamo-columnar) junction, testicular cancer, lymphoid leukemia, esophagus cancer, small intestine cancer, biliary tract cancer, cancer in digestive organs, or tumors thereof. In yet other embodiments, the method further comprises treatment of a cancer related to cystic fibrosis and the cancer related to cystic fibrosis is a lung cancer, a digestive tract cancer, colon cancer, or tumors thereof. In still other embodiments, the method further comprises treatment of a cancer related to cystic fibrosis and the cancer occurs after an organ transplantation. In certain embodiments, the method further comprises treatment of a cancer related to cystic fibrosis and the cancer related to cystic fibrosis is a tumor.

In some embodiments, the method further comprises one or more other cystic fibrosis treatments. In other embodiments, the method further comprises one or more other cystic fibrosis treatments and the other cystic fibrosis treatment comprises administering one or more of an antibiotic, an anti-inflammatory drug, or a mucus thinner. In still other embodiments, the method further comprises one or more other cystic fibrosis treatments and the other cystic fibrosis treatment comprises administering one or more non-drug respiratory therapies.

Some embodiments of the invention include method for treating a human for cystic fibrosis, comprising administering a composition comprising saracatinib, wherein the amount of saracatinib in the composition is no more than about 2.5 mg/kg human body weight. In some embodiments of the method, the amount of saracatinib in the composition is no more than about 1.5 mg/kg human body weight.

Other embodiments of the invention include a method for treating a human for cystic fibrosis, comprising administering a composition comprising saracatinib, VX770, and VX809. In some embodiments of the method, the amount of saracatinib in the composition is no more than about 1.5 mg/kg human body weight.

Some embodiments of the invention include a composition (e.g., a pharmaceutical composition) comprising saracatinib wherein the amount of saracatinib in the composition is no more than about 3.0 mg/kg human body weight, no more than about 2.5 mg/kg human body weight, no more than about 2.0 mg/kg human body weight, no more than about 1.5 mg/kg human body weight, or no more than about 1.0 mg/kg human body weight. The human body weight can be about 5 kg, about 25 kg, about 45 kg, about 60 kg, about 75 kg, about 85 kg, about 100 kg, about 200 kg, or from about 45 kg to about 85 kg.

Certain embodiments of the invention include a composition (e.g., a pharmaceutical composition) comprising saracatinib and (a) one or more correctors of ΔF508 CFTR (e.g., VX809 or VX661), (b) one or more potentiators of ΔF508 CFTR (e.g., VX770), or (c) both.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Western-blot data show Bands B (immature or ER form) and C (mature or membrane form) of CFTR

FIG. 2: Quantitation of Fluid Secretion by forskolin-induced swelling (FIS) for various compounds for Basal (−FSK) and +FSK.

FIG. 3: Fluid Secretion by forskolin-induced swelling (FIS) of human duodenal organoids (A) representative images (bar=20 micrometers) and (B) quantitation of fluid secretion.

FIG. 4: Fluid Secretion by forskolin-induced swelling (FIS) of mouse intestinal organoids (A) representative images (bar=20 micrometers) and (B) quantitation of fluid secretion.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include methods for treating an animal for cystic fibrosis comprising one or more administrations of one or more compositions comprising saracatinib. Other embodiments of the invention include treating an animal for cystic fibrosis comprising one or more administrations of one or more compositions comprising saracatinib, optionally a corrector of ΔF508 CFTR, and optionally a potentiator of ΔF508 CFTR. Still other embodiments of the invention include methods for treating a human with cystic fibrosis caused by the F508 deletion mutation in the cystic fibrosis transmembrane conductance regulator (CFTR), comprising one or more administrations of one or more compositions comprising saracatinib, and optionally VX770, VX809, or both. Additional embodiments of the invention are als

[symbol]

ΔF508 CFTR (also referred to as F508del CFTR) is the deletion of phenylaniline 508 (ΔF508 or F508del) of the cystic fibrosis transmembrane conductance regulator (CFTR).

Saracatinib (N-(5-chloro-1,3-benzodioxol-4-yl)-7-[2-(4-methyl-1-piperazinyl)ethoxy]-5-[(tetrahydro-2H-pyran-4-yl)oxy]-4-quinazolinamine; CAS No. 379231-04-6) is also known as AZD-0530. VX661 (1-(2,2-Difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide; CAS No. 1152311-62-0) is also known as tezacaftor. VX770 (N-(2,4-Di-tert-butyl-5-hydroxyphenyl)-4-oxo-1,4-dihydroquinoline-3-carboxamide; CAS No. 873054-44-5) is also known as ivacaftor or kalydeco. VX809 (3-{6-{[1-(2,2-Difluoro-1,3-benzodioxol-5-yl)cyclopropanecarbonyl]amino}-3-methylpyridin-2-yl}benzoic acid; CAS No. 936727-05-8) is also known as lumacaftor.

Treatments of Disease

Some embodiments of the invention include treatment of an animal with cystic fibrosis comprising administering saracatinib (and optionally VX770 and VX809). Administration to the animals can be accomplished by any number of suitable administration routes or formulations. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

In some embodiments, the amount of saracatinib administered to an animal (e.g., via a composition or a pharmaceutical composition) can be, but is not limited to about 0.005 mg/kg, about 0.01 mg/kg, about 0.05 mg/kg, about 0.1 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 10 mg/kg, about 20 mg/kg, about 30 mg/kg, about 40 mg/kg, about 50 mg/kg, about 80 mg/kg, about 100 mg/kg, about 150 mg/kg, no more than about 5.0 mg/kg, no more than about 4.5 mg/kg, no more than about 4.4 mg/kg, no more than about 4.3 mg/kg, no more than about 4.2 mg/kg, no more than about 4.1 mg/kg, no more than about 4.0 mg/kg, no more than about 3.9 mg/kg, no more than about 3.8 mg/kg, no more than about 3.7 mg/kg, no more than about 3.6 mg/kg, no more than about 3.5 mg/kg, no more than about 3.4 mg/kg, no more than about 3.3 mg/kg, no more than about 3.2 mg/kg, no more than about 3.1 mg/kg, no more than about 3.0 mg/kg, no more than about 2.9 mg/kg, no more than about 2.8 mg/kg, no more than about 2.7 mg/kg, no more than about 2.6 mg/kg, no more than about 2.5 mg/kg, no more than about 2.4 mg/kg, no more than about 2.3 mg/kg, no more than about 2.2 mg/kg, no more than about 2.1 mg/kg, no more than about 2.0 mg/kg, no more than about 1.9 mg/kg, no more than about 1.8 mg/kg, no more than about 1.7 mg/kg, no more than about 1.6 mg/kg, no more than about 1.5 mg/kg, no more than about 1.4 mg/kg, no more than about 1.3 mg/kg, no more than about 1.2 mg/kg, no more than about 1.1 mg/kg, no more than about 1.0 mg/kg, no more than about 0.9 mg/kg, no more than about 0.8 mg/kg, no more than about 0.7 mg/kg, no more than about 0.6 mg/kg, or no more than about 0.5 mg/kg animal body weight. The animal (e.g., human) body weight can be about 2 kg, about 5 kg, about 10 kg, about 15 kg, about 20 kg, about 25 kg, about 30 kg, about 35 kg, about 40 kg, about 45 kg, about 50 kg, about 55 kg, about 60 kg, about 65 kg, about 70 kg, about 75 kg, about 80 kg, about 85 kg, about 90 kg, about 95 kg, about 100 kg, about 150 kg, about 200 kg, from about 2 kg to about 200 kg, from about 10 kg to about 100 kg, from about 10 kg to about 85 kg, from about 45 kg to about 100 kg, or from about 45 kg to about 85 kg. These amounts (e.g., dosages) can be used as an effective amount or a therapeutically effective amount.

In some embodiments, treatment of an animal with cystic fibrosis comprises administering to the animal (a) saracatinib, (b) optionally one or more of a potentiator of ΔF508 CFTR (e.g., VX770), and (c) optionally one or more of a corrector of ΔF508 CFTR (e.g., VX661 or VX809). In other embodiments, treatment of an animal with cystic fibrosis comprises administering to the animal (a) saracatinib, (b) one or more of a potentiator of ΔF508 CFTR (e.g., VX770), and (c) optionally one or more of a corrector of ΔF508 CFTR (e.g., VX661 or VX809). In certain embodiments, treatment of an animal with cystic fibrosis comprises administering to the animal (a) saracatinib, (b) optionally one or more of a potentiator of ΔF508 CFTR (e.g., VX770), and (c) one or more of a corrector of ΔF508 CFTR (e.g., VX661 or VX809). In other embodiments, treatment of an animal with cystic fibrosis comprises administering to the animal (a) saracatinib, (b) one or more of a potentiator of ΔF508 CFTR (e.g., VX770), and (c) one or more of a corrector of ΔF508 CFTR (e.g., VX661 or VX809). In certain embodiments, VX660 and VX809 are in a combination drug (e.g., in the same pill), such as the brand name drug orkambi (also known as lumacaftor/ivacaftor); a single combination pill can be comprised of 200 mg of VX809 and 125 mg of VX770.

In some embodiments, a potentiator of ΔF508 CFTR can be any suitable molecule that is a potentiator of ΔF508 CFTR (e.g., a compound which normalizes or corrects (e.g., partially or completely) defective ΔF508 CFTR chloride channel gating), such as but not limited to PG-01, VX770, or tetrahydrobenzothiophene. In other embodiments, a corrector of ΔF508 CFTR can be any suitable molecule that is a corrector of ΔF508 CFTR (e.g., a compound which promotes or increases ΔF508 CFTR exit from the endoplasmic reticulum and/or accumulation in the plasma membrane), such as but not limited to Corr-4a, JY-29, CoPo-22, VRT-325, VX809, or VX661. The structures of the compounds PG-01, VX770, tetrahydrobenzothiophene, Corr-4a, JY-29, CoPo-22, VRT-325, and VX809 can be found in LUKACS et al., “CFTR: folding, misfolding and correcting the ΔF508 conformational defect” Trends Mol Med (2012) Vol. 18, No. 2, pp. 81-91 (which is herein incorporated by reference in its entirety). In certain embodiments, a compound can act as both a potentiator of ΔF508 CFTR and a corrector of ΔF508 CFTR.

In some embodiments, treatment of an animal with cystic fibrosis comprises administering to the animal saracatinib and optionally one or more of VX661, VX770, or VX809 (e.g., a composition comprising saracatinib and optionally one or more of VX661, VX770, or VX809). In other embodiments, treatment of an animal with cystic fibrosis comprises administering to the animal saracatinib and VX770 (e.g., a composition comprising saracatinib and VX770). In other embodiments, treatment of an animal with cystic fibrosis comprises administering to the animal saracatinib and VX661 (e.g., a composition comprising saracatinib and VX661). In other embodiments, treatment of an animal with cystic fibrosis comprises administering to the animal saracatinib and VX809 (e.g., a composition comprising saracatinib and VX809). In other embodiments, treatment of an animal with cystic fibrosis comprises administering to the animal saracatinib, VX770, and VX 809 (e.g., a composition comprising saracatinib, VX770, and VX809). In other embodiments, treatment of an animal with cystic fibrosis comprises administering to the animal saracatinib, VX770, and VX661 (e.g., a composition comprising saracatinib, VX770, and VX661).

The route of administration for treatment can be of any suitable route. Administration routes can be, but are not limited to the oral route, the parenteral route, the cutaneous route, the nasal route, the rectal route, the vaginal route, and the ocular route. In other embodiments, the administration route can be parenteral administration, a mucosal administration, intravenous administration, depot injection, subcutaneous administration, topical administration, intradermal administration, oral administration, sublingual administration, intranasal administration, or intramuscular administration. In some embodiments, the administration can be an intranasal administration, an aerosol administration, a nebulizer administration, a pressurized metered-dose inhaler (pMDI) administration, an inhaler administration, or a dry powder inhaler (DPI) administration. The choice of administration route can depend on the compound identity (e.g., the physical and chemical properties of the compound) as well as the age and weight of the animal, the particular disease (e.g., the type of cystic fibrosis), and the severity of the disease (e.g., stage or severity of disease). Of course, combinations of administration routes can be administered, as desired.

Some embodiments of the invention include a method for providing a subject with a composition comprising saracatinib described herein (e.g., a pharmaceutical composition) which comprises one or more administrations of one or more such compositions; the compositions may be the same or different if there is more than one administration.

Diseases that can be treated in an animal (e.g., mammals, porcine, canine, avian (e.g., chicken), bovine, feline, primates, rodents, monkeys, rabbits, mice, rats, and humans) using a composition comprising saracatinib (e.g., a composition comprising saracatinib, VX770, and VX809) include, but are not limited to cystic fibrosis.

In some embodiments, diseases that can be treated in an animal (e.g., mammals, porcine, canine, avian (e.g., chicken), bovine, feline, primates, rodents, monkeys, rabbits, mice, rats, and humans) using a composition comprising saracatinib (e.g., a composition comprising saracatinib, VX770, and VX809) include, but are not limited to cystic fibrosis diseases that include, but are not limited to cystic fibrosis caused by one or more mutations in the cystic fibrosis transmembrane conductance regulator (CFTR) or cystic fibrosis caused by the F508 deletion mutation in the cystic fibrosis transmembrane conductance regulator (CFTR).

In other embodiments, the method can further comprise treatment of a cancer related to cystic fibrosis (e.g., by a composition comprising saracatinib or a composition comprising saracatinib, VX770, and VX809). In other embodiments, the treatment of a cancer related to cystic fibrosis can comprise treatment of lung cancer, a digestive tract cancer, colon cancer, cancer at the cardio-esophageal junction, esophageal cancer, cancer at the gastro-esophageal (or squamo-columnar) junction, testicular cancer, lymphoid leukemia, esophagus cancer, small intestine cancer, biliary tract cancer, cancer in digestive organs, or tumors thereof. In certain embodiments, the treatment of a cancer related to cystic fibrosis can comprise treatment of lung cancer, a digestive tract cancer, colon cancer, or tumors thereof. In certain embodiments, the treatment of a cancer related to cystic fibrosis can comprise treatment of a cancer that occurs after an organ transplantation. In certain embodiments, the treatment of a cancer related to cystic fibrosis can comprise treatment of a cancerous tumor.

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease or cystic fibrosis).

In some embodiments, diseases that can be treated in an animal (e.g., mammals, porcine, canine, avian (e.g., chicken), bovine, feline, primates, rodents, monkeys, rabbits, mice, rats, and humans) using saracatinib (e.g., by a composition comprising saracatinib or a composition comprising saracatinib, VX770, and VX809) include, but are not limited to cystic fibrosis, cystic fibrosis caused by one or more mutations in the cystic fibrosis transmembrane conductance regulator (CFTR), or cystic fibrosis caused by the F508 deletion mutation in the cystic fibrosis transmembrane conductance regulator (CFTR).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating cystic fibrosis (e.g., cystic fibrosis caused by ΔF508 CFTR), treating can include but is not limited to prophylactic treatment and therapeutic treatment. As such, treatment can include, but is not limited to: preventing cystic fibrosis (e.g., cystic fibrosis caused by ΔF508 CFTR); reducing the risk of cystic fibrosis (e.g., cystic fibrosis caused by ΔF508 CFTR); ameliorating or relieving symptoms of cystic fibrosis (e.g., cystic fibrosis caused by ΔF508 CFTR); eliciting a bodily response against cystic fibrosis (e.g., cystic fibrosis caused by ΔF508 CFTR); inhibiting the development or progression of cystic fibrosis (e.g., cystic fibrosis caused by ΔF508 CFTR); inhibiting or preventing the onset of symptoms associated with cystic fibrosis (e.g., cystic fibrosis caused by ΔF508 CFTR); reducing the severity of cystic fibrosis (e.g., cystic fibrosis caused by ΔF508 CFTR); causing a regression of cystic fibrosis (e.g., cystic fibrosis caused by ΔF508 CFTR) or one or more of the symptoms associated with fibrosis (e.g., a decrease in the amount of fibrosis); causing remission of cystic fibrosis (e.g., cystic fibrosis caused by ΔF508 CFTR); or preventing relapse of cystic fibrosis (e.g., cystic fibrosis caused by ΔF508 CFTR). In some embodiments, treating does not include prophylactic treatment of cystic fibrosis (e.g., preventing or ameliorating future cystic fibrosis).

Treatment of an animal (e.g., human) can occur using any suitable administration method (such as those disclosed herein) and using any suitable amount of a saracatinib (e.g., by a composition comprising saracatinib or a composition comprising saracatinib, VX770, and VX809). In some embodiments, methods of treatment comprise treating an animal for cystic fibrosis (e.g., cystic fibrosis caused by ΔF508 CFTR). Some embodiments of the invention include a method for treating a subject (e.g., an animal such as a human or primate) with a composition comprising saracatinib (e.g., by a composition comprising saracatinib or a composition comprising saracatinib, VX770, and VX809) (e.g., a pharmaceutical composition) which comprises one or more administrations of one or more such compositions; the compositions may be the same or different if there is more than one administration.

In some embodiments, the method of treatment includes administering an effective amount of a composition comprising saracatinib (e.g., by a composition comprising saracatinib or a composition comprising saracatinib, VX770, and VX809). As used herein, the term “effective amount” refers to a dosage or a series of dosages sufficient to affect treatment (e.g., to treat cystic fibrosis, such as but not limited to cystic fibrosis caused by ΔF508 CFTR) in an animal and include dosages disclosed herein (e.g., those disclosed above). In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of saracatinib (e.g., by a composition comprising saracatinib or a composition comprising saracatinib, VX770, and VX809) (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 12 mg/kg, or about 15 mg/kg. The amount of a corrector of ΔF508 CFTR can be any amount disclosed herein (e.g., an amount disclosed in the previous sentences). The amount of a potentiator of ΔF508 CFTR can be any amount disclosed herein (e.g., an amount disclosed in the previous sentences). The amount of one or more of VX809, VX661, or VX770 can be any amount disclosed herein (e.g., an amount disclosed in the previous sentences). In regard to some embodiments, the dosage can be about 0.1 mg/kg human body weight, about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 10 mg/kg human body weight, about 50 mg/kg human body weight, about 80 mg/kg human body weight, or about 100 mg/kg human body weight. In some instances, an effective amount of saracatinib (e.g., by a composition comprising saracatinib or a composition comprising saracatinib, VX770, and VX809) (which can be administered to an animal such as mammals, rodents, mice, rabbits, feline, porcine, or canine) can be an amount of about 0.005 to about 50 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 20 mg/kg, about 30 mg/kg, about 40 mg/kg, about 50 mg/kg, about 80 mg/kg, about 100 mg/kg, or about 150 mg/kg. The amount of a corrector of ΔF508 CFTR can be any amount disclosed herein (e.g., an amount disclosed in the previous sentences). The amount of a potentiator of ΔF508 CFTR can be any amount disclosed herein (e.g., an amount disclosed in the previous sentences). The amount of one or more of VX809, VX661, or VX770 can be any amount disclosed herein (e.g., an amount disclosed in the previous sentences). In some embodiments, an effective amount of saracatinib (e.g., by a composition comprising saracatinib or a composition comprising saracatinib, VX770, and VX809) (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. The amount of a corrector of ΔF508 CFTR can be any amount disclosed herein (e.g., an amount disclosed in the previous sentences). The amount of a potentiator of ΔF508 CFTR can be any amount disclosed herein (e.g., an amount disclosed in the previous sentences). The amount of one or more of VX809, VX661, or VX770 can be any amount disclosed herein (e.g., an amount disclosed in the previous sentences). In regard to some conditions, the dosage can be about 0.1 mg/kg human body weight, about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 10 mg/kg human body weight, about 20 mg/kg human body weight, about 80 mg/kg human body weight, or about 100 mg/kg human body weight. The amount of a corrector of ΔF508 CFTR can be any amount disclosed herein (e.g., an amount disclosed in the previous sentences). The amount of a potentiator of ΔF508 CFTR can be any amount disclosed herein (e.g., an amount disclosed in the previous sentences). The amount of one or more of VX809, VX661, or VX770 can be any amount disclosed herein (e.g., an amount disclosed in the previous sentences). In some instances, an effective amount of saracatinib (e.g., by a composition comprising saracatinib or a composition comprising saracatinib, VX770, and VX809) (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 5.5 mg/kg, about 6 mg/kg, about 6.5 mg/kg, about 7.0 mg/kg, about 7.5 mg/kg, about 8.0 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. The amount of a corrector of ΔF508 CFTR can be any amount disclosed herein (e.g., an amount disclosed in the previous sentences). The amount of a potentiator of ΔF508 CFTR can be any amount disclosed herein (e.g., an amount disclosed in the previous sentences). The amount of one or more of VX809, VX661, or VX770 can be any amount disclosed herein (e.g., an amount disclosed in the previous sentences).

“Therapeutically effective amount” means an amount effective to achieve a desired and/or beneficial effect (e.g., decreasing amount of cystic fibrosis). A therapeutically effective amount can be administered in one or more administrations. For some purposes of this invention, a therapeutically effective amount is an amount appropriate to treat an indication (e.g., to treat cystic fibrosis). By treating an indication is meant achieving any desirable effect, such as one or more of palliate, ameliorate, stabilize, reverse, slow, or delay disease (e.g., cystic fibrosis) progression, increase the quality of life, or to prolong life. Such achievement can be measured by any suitable method, such as but not limited to measurement of the extent of secretion, normalization of CFTR (or a CFTR mutant) chloride channel gating, promoting CFTR (or a CFTR mutant) exit from the endoplasmic reticulum, promoting CFTR (or a CFTR mutant) accumulation in the plasma membrane, lung weight, body weight, lung function, or any suitable method to assess the progression of cystic fibrosis.

In some embodiments, other fibrosis treatments are optionally included, and can be used with the inventive treatments described herein (e.g., administering saracatinib (e.g., by a composition comprising saracatinib or a composition comprising saracatinib, VX770, and VX809)). Other fibrosis treatments can comprise any known fibrosis treatment or cystic fibrosis treatment that is suitable to treat cystic fibrosis. Examples of known fibrosis or cystic fibrosis treatments include but are not limited to administration of: antibiotics (e.g., penicillins, methicillin, oxacillin, nafcillin, cabenicillin, ticarcillin, piperacillin, mezlocillin, azlocillin, ticarcillin clavulanic acid, piperacillin tazobactam, cephalosporins, cephalexin, cefdinir, cefprozil, cefaclor, cefepime, sulfa, sulfamethoxazole, trimethoprim, erythromycin/sulfisoxazole, macrolides, erythromycin, clarithromycin, azithromycin, tetracyclines, tetracycline, doxycycline, minocycline, tigecycline, vancomycin, imipenem, meripenem, colistimethate/colistin, aminoglycosides, tobramycin, amikacin, gentamicin, quinolones, aztreonam, or linezolid), anti-inflammatory drugs (e.g., NSAIDs, aspirin, ibuprofen, naproxen, corticosteroids, cortisol, corticosterone, cortisone, or aldosterone), bronchodilators (e.g., albuterol or levalbuterol hydrochloride), or mucus thinners (e.g., hypertonic saline or Dornase alfa). Other fibrosis or cystic fibrosis treatments can also include administering a non-drug respiratory therapy such as but not limited to airway clearance techniques (e.g., postural drainage and chest percussion, exercise, breathing exercises, or use of mechanical equipment such as high-frequency chest compression vest or positive expiratory pressure therapy). Other fibrosis or cystic fibrosis treatments can also include organ transplantation (e.g., lung, skin, kidney, liver, or heart).

In some embodiments, administration of one or more of a ΔF508 CFTR potentiator, a ΔF508 CFTR corrector, VX809, VX661, or VX770 can be used as part of the treatment regime (i.e., in addition to administration of saracatinib and as an other cystic fibrosis treatment); administration of one or more of a ΔF508 CFTR potentiator, a ΔF508 CFTR corrector, VX809, VX661, or VX770, can include separate administrations (i.e., in a separate composition from saracatinib) or can be added to the composition comprising saracatinib.

In some embodiments, additional optional treatments (e.g., as an other fibrosis or cystic fibrosis treatment) can also include one or more of surgical intervention, hormone therapies, immunotherapy, adjuvant systematic therapies, and cancer therapies (e.g., radiation treatment, chemotherapies, cancer immunotherapies, or any suitable cancer treatment).

Compositions Used for Treating

In some embodiments, saracatinib, the ΔF508 CFTR potentiator, the ΔF508 CFTR corrector, VX809, VX661, or VX770 can be in the form of a salt, an ester, or a solvate. In other embodiments, saracatinib, the ΔF508 CFTR potentiator, the ΔF508 CFTR corrector, VX809, VX661, or VX770 can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). Esters can include any suitable esters such as but not limited to when an —OH group is replaced by an —O-alkyl group, where alkyl can be but is not limited to methyl, ethyl, propyl, or butyl. Solvates can include any suitable solvent (e.g., water, alcohols, ethanol) complexed (e.g., reversibly associated) with the molecule (e.g., opioid receptor inhibitor).

In certain embodiments, saracatinib, the ΔF508 CFTR potentiator, the ΔF508 CFTR corrector, VX809, VX661, or VX770 can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, saracatinib, the ΔF508 CFTR potentiator, the ΔF508 CFTR corrector, VX809, VX661, or VX770 can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising saracatinib and optionally one or more of a ΔF508 CFTR potentiator, a ΔF508 CFTR corrector, VX809, VX661, or VX770. In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

“Therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of the lung weight, body weight, lung function, extent of secretion, normalization of CFTR (or a CFTR mutant) chloride channel gating, promoting CFTR (or a CFTR mutant) exit from the endoplasmic reticulum, promoting CFTR (or a CFTR mutant) accumulation in the plasma membrane, or any suitable method to assess the progression of cystic fibrosis.

In some embodiments, saracatinib, the ΔF508 CFTR potentiator, the ΔF508 CFTR corrector, VX809, VX661, or VX770 can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, depot injection (e.g., solid or oil based), subcutaneous administration, topical administration, intradermal administration, oral administration, sublingual administration, intranasal administration, or intramuscular administration. In other embodiments, the pharmaceutical composition can be presented in a dosage form which is suitable for an intranasal administration, an aerosol administration, a nebulizer administration, a pressurized metered-dose inhaler (pMDI) administration, an inhaler administration, or a dry powder inhaler (DPI) administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. For example, an intranasal administration, an aerosol administration, a nebulizer administration, a pressurized metered-dose inhaler (pMDI) administration, an inhaler administration, or a dry powder inhaler (DPI) administration, could include one or more formulary ingredients.

In certain embodiments, pharmaceutical compositions can be formulated to release saracatinib, the ΔF508 CFTR potentiator, the ΔF508 CFTR corrector, VX809, VX661, or VX770 substantially immediately upon the administration or any substantially predetermined time or time after administration. Such formulations can include, for example, controlled release formulations such as various controlled release compositions and coatings. For example, an intranasal administration, an aerosol administration, a nebulizer administration, a pressurized metered-dose inhaler (pMDI) administration, an inhaler administration, or a dry powder inhaler (DPI) administration, could be used for a controlled release (e.g., of saracatinib), and in some instances, could be administered once per hour (or once per day, several times per day, more than once per day, once per week, several times per week, once per three months, once per six months, or once per year).

Other formulations (e.g., formulations of a pharmaceutical composition) can, in certain embodiments, include those incorporating the drug (or control release formulation) into food, food stuffs, feed, or drink. For example, saracatinib (and optionally one or more of a ΔF508 CFTR potentiator, a ΔF508 CFTR corrector, VX809, VX661, or VX770) could be administered orally once per day, twice per day, three times per day, more than once per day, once per two days, or once per week.

Some embodiments of the invention can include methods of treating an organism for cystic fibrosis. In certain embodiments, treating comprises administering saracatinib (and optionally one or more of a ΔF508 CFTR potentiator, a ΔF508 CFTR corrector, VX809, VX661, or VX770). In other embodiments, treating comprises administering saracatinib (and optionally one or more of a ΔF508 CFTR potentiator, a ΔF508 CFTR corrector, VX809, VX661, or VX770) to an animal that is effective to treat cystic fibrosis. In some embodiments, a composition or pharmaceutical composition comprises saracatinib (and optionally one or more of a ΔF508 CFTR potentiator, a ΔF508 CFTR corrector, VX809, VX661, or VX770) which can be administered to an animal (e.g., mammals, primates, monkeys, or humans) in an amount of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight, about 1.0 mg/kg human body weight, about 1.5 mg/kg human body weight, about 2.0 mg/kg human body weight, about 2.5 mg/kg human body weight, about 3.0 mg/kg human body weight, about 3.5 mg/kg human body weight, about 4.0 mg/kg human body weight, about 4.5 mg/kg human body weight, about 5.0 mg/kg human body weight, about 6.5 mg/kg human body weight, about 10 mg/kg human body weight, about 50 mg/kg human body weight, about 80 mg/kg human body weight, or about 100 mg/kg human body weight. In some instances, some animals (e.g., mammals, mice, rabbits, feline, porcine, or canine) can be administered a dosage of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, about 5.0 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, saracatinib (and optionally one or more of a ΔF508 CFTR potentiator, a ΔF508 CFTR corrector, VX809, VX661, or VX770) can be administered in combination with one or more other therapeutic agents to treat a given cystic fibrosis.

In some embodiments, the compositions can include a unit dose of one or more saracatinib (and optionally one or more of a ΔF508 CFTR potentiator, a ΔF508 CFTR corrector, VX809, VX661, or VX770) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

The presently-disclosed subject matter is further illustrated by the following specific but non-limiting examples. The following examples may include compilations of data that are representative of data gathered at various times during the course of development and experimentation related to the present invention.

### Examples

Materials and Methods

Compounds

Saracatinib (N-(5-chloro-1,3-benzodioxol-4-yl)-7-[2-(4-methyl-1-piperazinyl)ethoxy]-5-[(tetrahydro-2H-pyran-4-yl)oxy]-4-quinazolinamine; CAS No. 379231-04-6; also known as AZD-0530) was purchased from Tocris Bioscience.

VX809 (3-{6-{[1-(2,2-Difluoro-1,3-benzodioxol-5-yl)cyclopropanecarbonyl]amino}-3-methylpyridin-2-yl}benzoic acid; CAS No. 936727-05-8; also known as lumacaftor) was purchased from Selleck Chemicals.

VX770 (N-(2,4-Di-tert-butyl-5-hydroxyphenyl)-4-oxo-1,4-dihydroquinoline-3-carboxamide; CAS No. 873054-44-5; also known as ivacaftor or kalydeco) was purchased from Selleck Chemicals.

VX661 (1-(2,2-Difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide; CAS No. 1152311-62-0; also known as tezacaftor) was purchased from Selleck Chemicals.

Enteroid Cultures

Mouse intestinal organoids were cultured as described previously (DALTON et al., “New model of cystic fibrosis transmembrane conductance regulator proposes active channel-lik [symbol] Inf Model (2012) Vol. 52, pp. 1842-1853). Human duodenal crypt isolation and enteroid expansion was performed as described previously (<<https://www.jove.com/video/52483/establishment-human-epithelial-enteroids-colonoids-from-whole-tissue>>) with some adaptations. Briefly, fresh biopsy is rinsed in ice-cold Dulbecco's Phosphate buffered saline without Ca²⁺ and Mg²⁺ (DPBS, Gibco), mounted and immersed in DPBS in a silica gel coated petri-dish using minutien pins with the mucosal side facing up.

Mucosa is gently scraped with curved forceps to remove villi and debris followed by 3-4 washes with DPBS. Crypts were dissociated using 2 mM EDTA (30 min, 4° C. with gentle shaking) followed by gentle scraping of the mucosa. The crypt suspension was filtered through a 150 μm nylon mesh twice and pelleted at 50×g, 4° C. The crypt pellet was resuspended in matrigel matrix (200 to 500 crypts/50 μl matrigel per well of a 24 well plate). Matrigel was allowed to polymerize by placing the plate in a 37° C., 5% CO₂ incubator for 30 min followed by addition of complete growth factor supplemented human minigut medium (Advanced DMEM/F12 medium with 2 mM glutamine, 10 mM HEPES, 100 U/mL penicillin, 100 g/mL streptomycin, 1 N2 supplement, 1 B27 supplement and 1% BSA supplemented with 50% Wnt-3A-conditioned medium, 1 μg/ml R-spondin 1, 100 ng/ml Noggin. 50 ng/mL EGF, 500 nM A-83-01, 10 μM SB202190, 10 nM [Leu]15-Gastrin 1, 10 mM Nicotinamide and 1 mM N-Acetylcysteine.

Fluid Secretion Measurement in Intestinal Spheroids

Isolation of intestinal spheres and measurement of fluid secretion were performed as described previously (MOON et al., “Compartmentalized accumulation of cAMP near complexes of multidrug resistance protein 4 (MRP4) and cystic fibrosis transmembrane conductance regulator (CFTR) contributes to drug-induced diarrhea.” J Biol Chem (2015) Vol. 290, pp. 11246-11257). Day 1-4 intestinal organoids were treated with 0.1-10 μM of the test compound for 24 h before stimulation of CFTR function using forskolin (10 μM). Fluid secretion measurements were done before and after 30 min of a stimulation period for mouse organoids and 120 min for human organoids. Quantitation of fluid secretion in the intestinal spheres was performed as described previously (MOON et al., “Compartmentalized accumulation of cAMP near complexes of multidrug resistance protein 4 (MRP4) and cystic fibrosis transmembrane conductance regulator (CFTR) contributes to drug-induced diarrhea.” J Biol Chem (2015) Vol. 290, pp. 11246-11257; DEKKERS et al., “A functional CFTR assay using primary cystic fibrosis intestinal organoids” Nature Medicine (2013) Vol. 19, pp. 939-945).

Results and Discussion

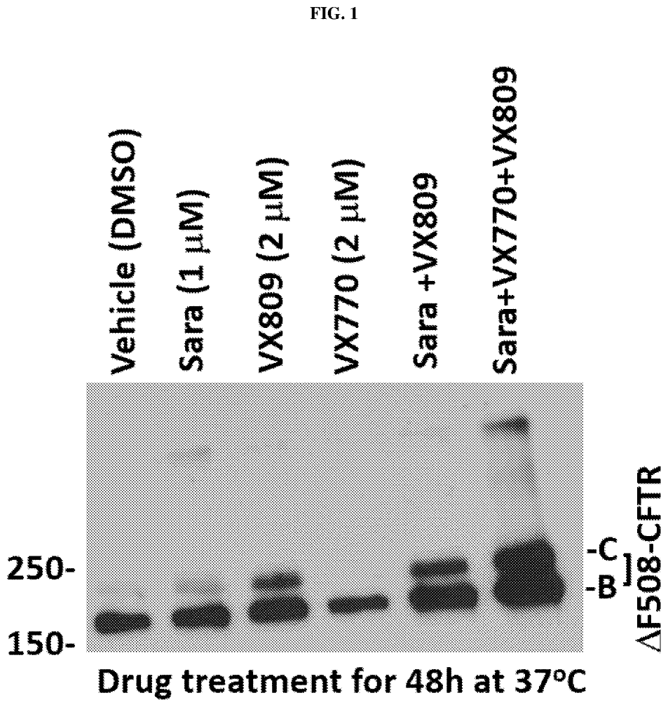

FIG. 1—Western-blot data show Bands B (immature or ER form) and C (mature or membrane form) of CFTR. Treatment of F508del CFTR expressing HEK 293 cells with the indicated combination of compounds. For “Sara+VX809” lane and the “Sara+VX770+VX809” lane, the concentrations were as follows: saracatinib was 0.1, VX809 was 2 micromolar, and VX770 was 2 micromolar. By monitoring the processing of F508del CFTR, we showed that F508del CFTR trafficking was rescued. Additionally, a synergistic effect was observed upon simultaneous treatment of cells with saracatinib with VX-809 and especially with saracatinib with VX-809 and VX770.

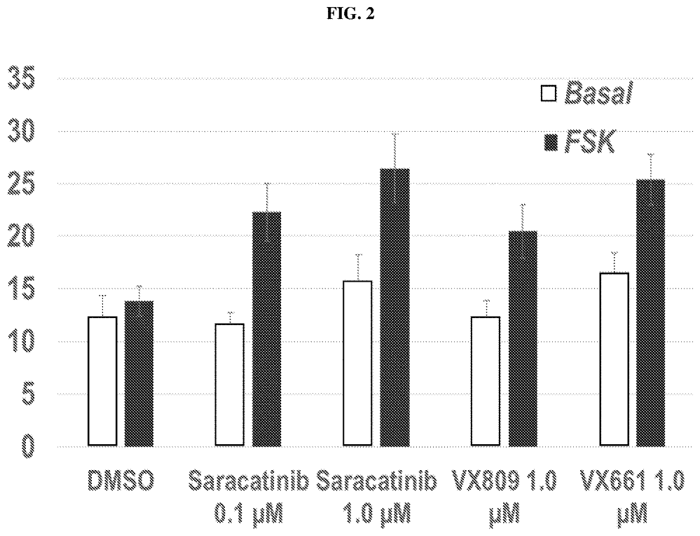

FIG. 2—Quantitation of Fluid Secretion by forskolin-induced swelling (FIS) for various compounds for Basal (−FSK) and +FSK Quantitation of fluid secretion (forskolin-induced swelling—FIS) in intestinal organoids (F508del CFTR homozygous CF patient) demonstrating fluid secretion in response to the test compounds.

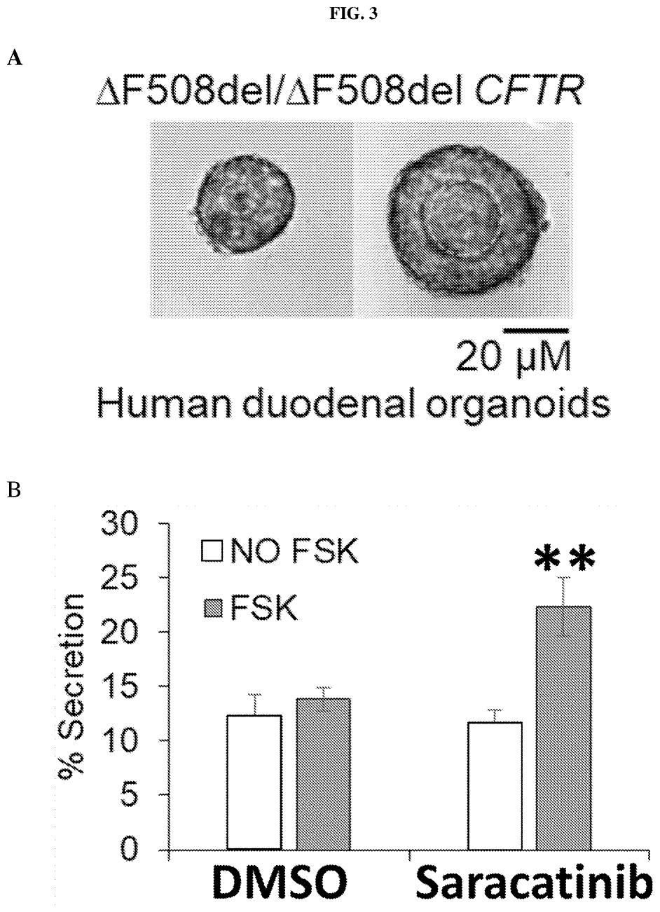

Fluid Secretion by forskolin-induced swelling (FIS) of human duodenal organoids (A) representative images (bar=20 micrometers) and (B) quantitation of fluid secretion. In FIG. 3A, the representative images show fluid secretion with FIS in response to 0.1 micromolar saracatinib and indicates that fluid secretion occurs when saracatinib is added (FIG. 3A right), but secretion does not occur when saracatinib is not added (FIG. 3A left). In FIG. 3B, the bar graph shows quantitation of secretion and indicates that fluid secretion occurs when saracatinib is added, but secretion does not occur when saracatinib is not added. The ** indicates that the difference in FSK vs no FSK in saracatinib treated organoids is significant with a p value of 0.01.

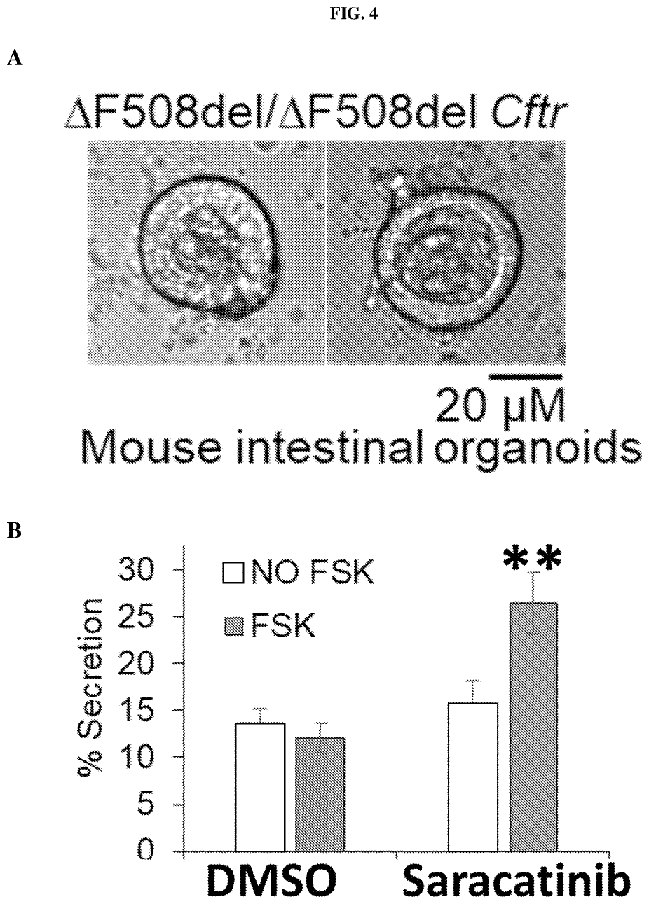

Fluid Secretion by forskolin-induced swelling (FIS) of mouse intestinal organoids (A) representative images (bar=20 micrometers) and (B) quantitation of fluid secretion. In FIG. 4A, the representative images show fluid secretion with FIS in response to 0.1 micromolar saracatinib and indicates that fluid secretion occurs when saracatinib is added (FIG. 4A right), but secretion does not occur when saracatinib is not added (FIG. 4A left). In FIG. 4B, the bar graph shows quantitation of secretion and indicates that fluid secretion occurs when saracatinib is added, but secretion does not occur when saracatinib is not added. The ** indicates that the difference in FSK vs no FSK in saracatinib treated organoids is significant with a p value of 0.01.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner Indeed, various modifications of the invention in addition to those described herein will become apparent to those skilled in the art from the foregoing description and the accompanying figures. Such modifications are intended to fall within the scope of the appended claims.

What is claimed is:

1. A method for treating an animal for cystic fibrosis, comprising one or more administrations of one or more compositions comprising saracatinib, wherein the compositions may be the same or different if there is more than one administration.

2. The method of claim 1, wherein at least one of the one or more compositions further comprises a corrector of ΔF508 CFTR, a potentiator of ΔF508 CFTR, or both.

3. The method of claim 1 or claim 2, wherein at least one of the one or more compositions further comprises one or more of VX809, VX661, or VX770.

4. The method of any of claims 1-3, wherein at least one of the one or more compositions further comprises VX770.

5. The method of any of claims 1-4, wherein at least one of the one or more compositions further comprises VX809.

6. The method of any of claims 1-5, wherein at least one of the one or more compositions further comprises VX661.

7. The method of any of claims 1-6, wherein at least one of the one or more compositions further comprises VX770 and VX809.

8. The method of any of claims 1-7, wherein the amount of saracatinib in at least one of the one or more compositions is from about 0.0001% (by weight total composition) to about 99%.

9. The method of any of claims 1-8, wherein the amount of saracatinib in at least one of the one or more compositions is no more than about 3.0 mg/kg.

10. The method of any of claims 1-9, wherein the amount of saracatinib in at least one of the one or more compositions is no more than about 2.0 mg/kg.

11. The method of any of claims 1-10, wherein at least one of the one or more compositions further comprises a formulary ingredient.

12. The method of any of claims 1-11, wherein at least one of the one or more compositions is a pharmaceutical composition.

13. The method of any of claims 1-12, wherein at least one of the one or more administrations comprises parenteral administration, a mucosal administration, intravenous administration, depot injection, subcutaneous administration, topical administration, intradermal administration, oral administration, sublingual administration, intranasal administration, or intramuscular administration.

14. The method of any of claims 1-13, wherein at least one of the one or more administrations comprises an intranasal administration, an aerosol administration, a nebulizer administration, a pressurized metered-dose inhaler (pMDI) administration, an inhaler administration, or a dry powder inhaler (DPI) administration.

15. The method of any of claims 1-14, wherein if there is more than one administration at least one composition used for at least one administration is different from the composition of at least one other administration.

16. The method of any of claims 2-15, wherein one or more of VX809, VX661, or VX770 in at least one of the one or more compositions is administered to the animal in an amount of from about 0.005 mg/kg animal body weight to about 100 mg/kg animal body weight.

17. The method of any of claims 1-16, wherein the animal is a human, a rodent, or a primate.

18. The method of any of claims 1-17, wherein the animal is in need of treatment of cystic fibrosis.

19. The method of any of claims 1-18, wherein the method comprises treatment of cystic fibrosis caused by one or more mutations in the cystic fibrosis transmembrane conductance regulator (CFTR).

20. The method of any of claims 1-19, wherein the method comprises treatment of cystic fibrosis caused by the F508 deletion mutation in the cystic fibrosis transmembrane conductance regulator (CFTR).

21. The method of any of claims 1-20, wherein the method further comprises treatment of a cancer related to cystic fibrosis.

22. The method of any of claims 1-21, wherein the method further comprises treatment of a cancer related to cystic fibrosis and the cancer related to cystic fibrosis is a lung cancer, a digestive tract cancer, colon cancer, cancer at the cardio-esophageal junction, esophageal cancer, cancer at the gastro-esophageal (or squamo-columnar) junction, testicular cancer, lymphoid leukemia, esophagus cancer, small intestine cancer, biliary tract cancer, cancer in digestive organs, or tumors thereof.

23. The method of any of claims 1-22, wherein the method further comprises treatment of a cancer related to cystic fibrosis and the cancer related to cystic fibrosis is a lung cancer, a digestive tract cancer, colon cancer, or tumors thereof.

24. The method of any of claims 1-23, wherein the method further comprises treatment of a cancer related to cystic fibrosis and the cancer occurs after an organ transplantation.

25. The method of any of claims 1-24, wherein the method further comprises treatment of a cancer related to cystic fibrosis and the cancer related to cystic fibrosis is a tumor.

26. The method of any of claims 1-25, wherein the method further comprises one or more other fibrosis treatments.

27. The method of any of claims 1-26, wherein the method further comprises one or more other fibrosis treatments and the other fibrosis treatment comprises administering one or more of an antibiotic, an anti-inflammatory drug, or a mucus thinner.

28. The method of any of claims 1-27, wherein the method further comprises one or more other cystic fibrosis treatments and the other cystic fibrosis treatment comprises administering one or more non-drug respiratory therapies.

29. A method for treating a human for cystic fibrosis, comprising administering a composition comprising saracatinib, wherein the amount of saracatinib in the composition is no more than about 2.5 mg/kg human body weight.

30. The method of claim 29, wherein the amount of saracatinib in the composition is no more than about 1.5 mg/kg human body weight.

31. A method for treating a human for cystic fibrosis, comprising administering a composition comprising saracatinib, VX770, and VX809.

32. The method of claim 31, wherein the amount of saracatinib in the composition is no more than about 1.5 mg/kg human body weight.

33. A composition (e.g., a pharmaceutical composition) comprising saracatinib wherein the amount of saracatinib in the composition is no more than about 3.0 mg/kg human body weight, no more than about 2.5 mg/kg human body weight, no more than about 2.0 mg/kg human body weight, no more than about 1.5 mg/kg human body weight, or no more than about 1.0 mg/kg human body weight.

34. A composition (e.g., a pharmaceutical composition) comprising saracatinib and (a) one or more correctors of ΔF508 CFTR (e.g., VX809 or VX661), (b) one or more potentiators of ΔF508 CFTR (e.g., VX770), or (c) both.
